(19) United States
(12) Patent Application Publication
(10) Pub. No.: US 2005/0192675 A1
(43) Pub. Date: Sep. 1, 2005

(54) Device for use in stimulating bone growth

(76) Inventor: Dane Q. Robinson, Mesa, AZ (US)

Correspondence Address: SQUIRE, SANDERS & DEMPSEY .L.L.P.;Two Renaissance Square, Suite 2700, 40 North Central Avenue, Phoenix, AZ, 85004-4498, US

(21) Appl. No.: 10/791,661

(22) Filed: Mar. 1, 2004

(51) Int. Cl. A61F 2/28; A61C 8/00

(52) U.S. Cl. 623/23.46; 623/23.54; 623/17.17; 433/174

(57) ABSTRACT

A method of growing jaw bone utilizing a device that can receive a sponge (or other material) that includes or is a substance that stimulates bone growth. The device positions the sponge next to the bone to be regenerated and is preferably a wire mesh partially surrounding the bone to be regenerated. The device defines a cavity having at least one or more openings through which the material may be inserted and thus received in the cavity.

### DESCRIPTION OF THE INVENTION

1. Field of the Invention

The present invention relates to a device for assisting in regenerating bone, most particularly jaw bone, by creating a cavity into which a material may be received and positioned next to the bone to be regenerated, wherein the material preferably includes a substance that stimulates bone growth.

2. Background of the Invention

The placement of endosseous dental implants has been well documented for over 30 years; however, the success of endosseous dental implants may be limited by the quality and quantity of existing bone a given patient presents. Due to the destructive nature of dentures to underlying jaw bone as well as to the fact that bone is not internally stimulated by tooth roots, many people have a limited amount of bone for the placement of dental implants, especially those who have been missing teeth for an extended period.

Bone grafting is one method for treating those without enough bone to accept dental implants. Some methods of bone grafting include (1) affixing blocks of hip bone to the jaw, and (2) using freeze-dried demineralized bone protein as a stimulant to cause the patient's jaw bone cells to become active and produce new bone in the existing bone areas and into the new bone graft areas. Through experience and research, it has become evident that, for bone grafting to be successful, it must be given an isolated space to grow, protected from muscular pressure, tissue impingement and chewing forces. In order to create this space, many approaches have been proposed. For example, both Syers (U.S. Pat. No. 5,297,563) and Magnusson et al (U.S. Pat. No. 4,961,707) teach the use of a fabric-like membrane which is used over a bony defect. Although this barrier creates a space isolated from the invasion of epithelial cells into the bony defect or bone graft area, it does not create a space protected from chewing forces or tissue pressure.

Morgan (U.S. Pat. No. 5,380,328) teaches the use of a composite perforated titanium mesh layered with polytetrafluoraethylene (PTFE or TEFLON) fibers. Even though this approach would be feasible for creating a protected space in which bone could grow, it has some severe limitations. This material requires the placement of peripheral bone screws into the edges of the mesh in order to directly fix the titanium mesh to the jaw bone, and then bending or tenting the center area of the mesh in order to create the protected space. Often, it is not feasible to place the peripheral bone screws in the peripheral areas for fear of damage to the inferior alveolar nerves or sinus penetration or damage to nearby tooth roots. The protrusion of these screws above the mesh is also of concern as potentially causing tissue irritation.

U.S. Pat. No. 6,238,214 to Robinson, the disclosure of which is incorporated herein by reference, teaches a tissue regeneration plate for creating a space relatively free of pressure, thus allowing jaw bone to grow into the space.

It is also known that bone growth is stimulated by morphogenetic proteins. Published U.S. patent application No. U.S. 2002/0082694 A1 entitled “Highly-Mineralized Osteogenic Sponge Compositions, and Uses Thereof,” the disclosure of which is incorporated herein by reference, teaches osteogenic sponge compositions including an osteogenic factor, preferably one or more of bone morphogenetic protein (BMP) or LIM mineralization protein (LMP), which stimulates bone growth. The osetoinductive factor preferably stimulates the production or activity of osteoblasts and/or osteoclasts to stimulate bone growth. The sponge matrix is preferably collogenous and a wide variety of collogen materials may be used.

### SUMMARY OF THE INVENTION

The invention is a device to assist in growing jaw bone. The device includes a cavity that preferably creates a space protecting an area in which it is desired to regenerate bone. The cavity can receive a material (such as a sponge) that includes (or is) a substance that stimulates bone growth (such as BMP and/or LMP) and positions the material next to the bone to be regenerated. The device is preferably a wire mesh that can partially surround the bone to be regenerated and the device defines a cavity having at least one or more openings through which the material may be inserted and thus received in the cavity.

### BRIEF DESCRIPTION OF THE DRAWINGS

FIG. 1 is a device according to the invention.

FIG. 2 shows an alternate device according to the invention.

FIG. 3 shows two devices according to the invention in actual size.

FIGS. 4A-4F are dental casts showing generally some various positions on a jaw where a device according to the invention could be positioned.

FIG. 5 is a side view of a device according to the invention mounted on a mating component that can be affixed to a post in a jaw bone.

FIG. 6 is a top view of the device of FIG. 5.

FIG. 7A is a top, perspective view of a preformed device according to the invention.

FIG. 7B is a bottom, perspective view of the device of FIG. 7A.

FIG. 7C is a top, perspective view of a another preformed device according to the invention.

FIG. 7D is a bottom, perspective view of the device of FIG. 7C.

FIG. 8 shows a jaw bone with posts inserted into the bone.

FIG. 9 shows a device according to the invention mounted on one of the posts shown in FIG. 8.

FIG. 10 shows a sponge saturated with morphegenetic protein being inserted into the cavity of the device of FIG. 9.

### DETAILED DESCRIPTION OF PREFERRED EMBODIMENTS

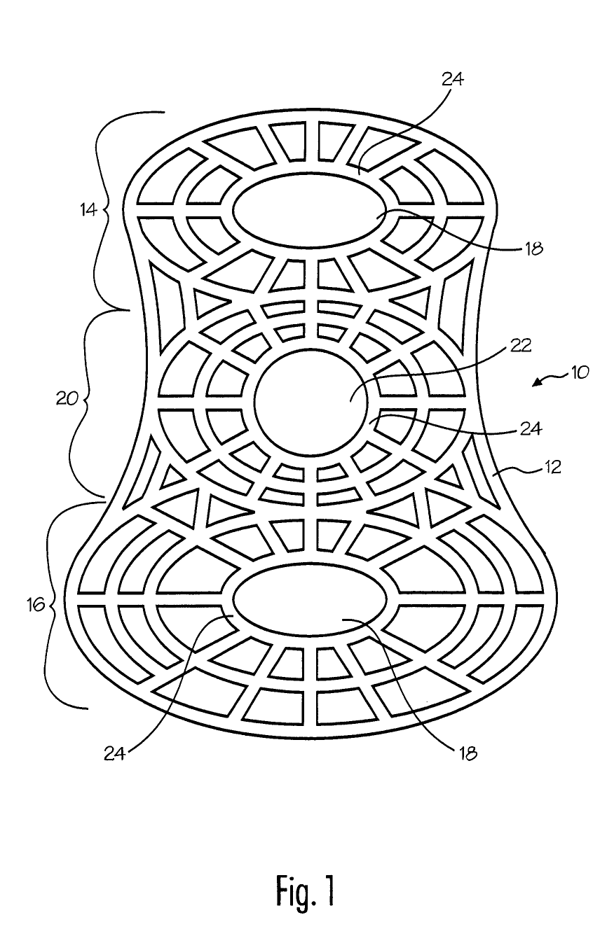

Turning now to the drawings, where the purpose is to describe preferred embodiments of the invention and not to limit same, FIG. 1 shows a device 10 according to the invention.

Device 10 as shown is a wire mesh structure preferably formed primarily of 0.015″ diameter wire 12, which is preferably steel. However, device 10 can be formed of any material suitable for being retained in the mouth for an extended period and that can generally maintain its shape while in use. Device 10, or any device according to the invention, may be made of any suitable plastic or metal, and may be of any size or shape suitable for retaining a material (as defined below) next to a bone to be regenerated. Further, device 10 or any device according to the invention can comprise material that can be absorbed by the body, such materials being generally known.

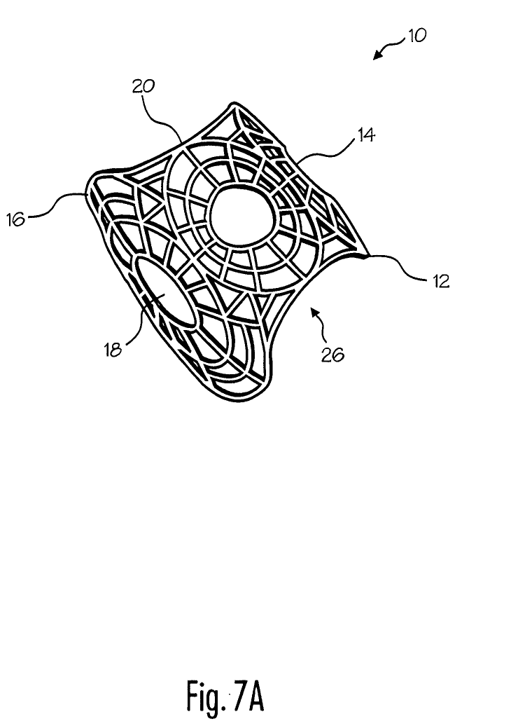

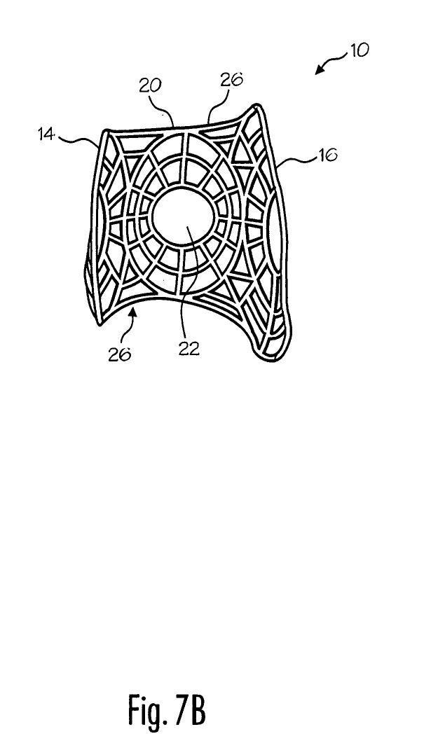

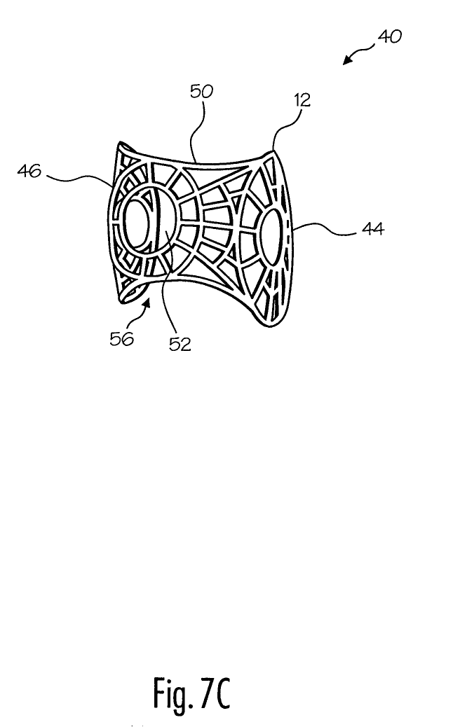

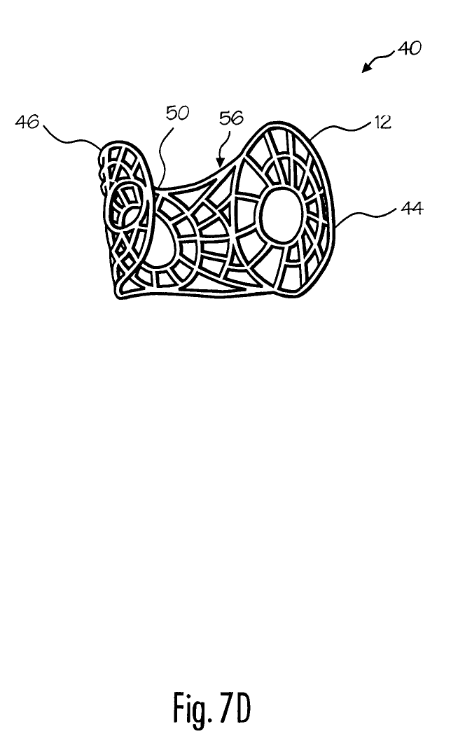

Device 10 has two sides 14 and 16, as best seen in FIGS. 1, 7A and 7B, has openings 18, one in side 14 and another in side 16. Each opening 18 is dimensioned so that a material may be inserted therethrough and each opening 18 may be of any size and shape suitable for this purpose. As shown, openings 18 are preferably oval and about 0.180″ by 0.090″, but any suitable dimension may be used. Moreover, each opening need not be the same size. Device 10 further includes a top region 20 having an aperture 22 defined by wire 24, which is preferably 0.020″ in diameter. As shown in FIGS. 7A and 7B, device 10 is formed (by bending using any suitable technique) to fit onto an area of jawbone, at which time a cavity 26 is defined by sides 14, 16 and top region 20.

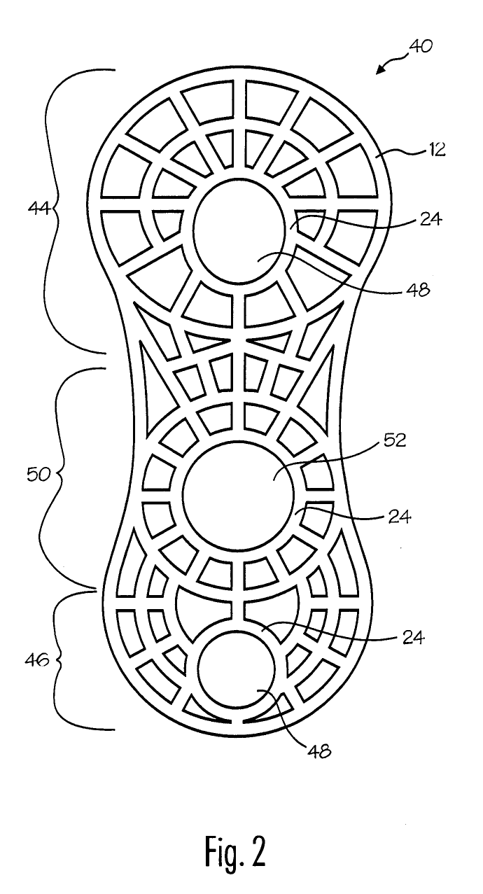

FIG. 2 shows a device 40 for use in a jaw bone, preferably for a tooth other than a molar, such as an incisor or bicuspid. Device 40 can be formed of any material that is used to form device 10, and as shown is a mesh structure formed of wires 12 and 22. Device 40 is shown as flat, prior to being shaped for use. Device 40 as shown is made of a wire mesh 42 and has sides 44, 46 with an opening 48 in each side. A top portion 50 includes an opening 52 that is defined by a wire 54 having a diameter of 0.020″. However, device 10 can be formed of any material suitable for being retained in the mouth for an extended period and that can generally maintain its shape while in use.

Device 40 has two sides 44 and 46, as best seen in FIGS. 2, 7C and 7D, has openings 48, one in side 44 and another in side 46. Each opening 48 is dimensioned so that a material may be inserted therethrough and each opening 48 may be of any size and shape suitable for this purpose. Moreover, each opening 48 need not be the same size. Device 40 further includes a top region 50 having an aperture 52 defined by wire 54, which is preferably 0.020″ in diameter. As shown in FIGS. 7C and 7D, device 40 is formed (by bending using any suitable technique) to fit onto an area of jawbone, at which time a cavity 56 is defined by sides 44, 46 and top portion 50.

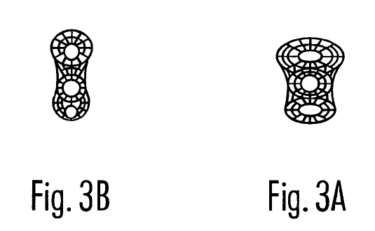

FIGS. 3A and 3B show, respectively, devices 10 and 40, essentially in actual size prior to being formed to fit onto an area of a jaw.

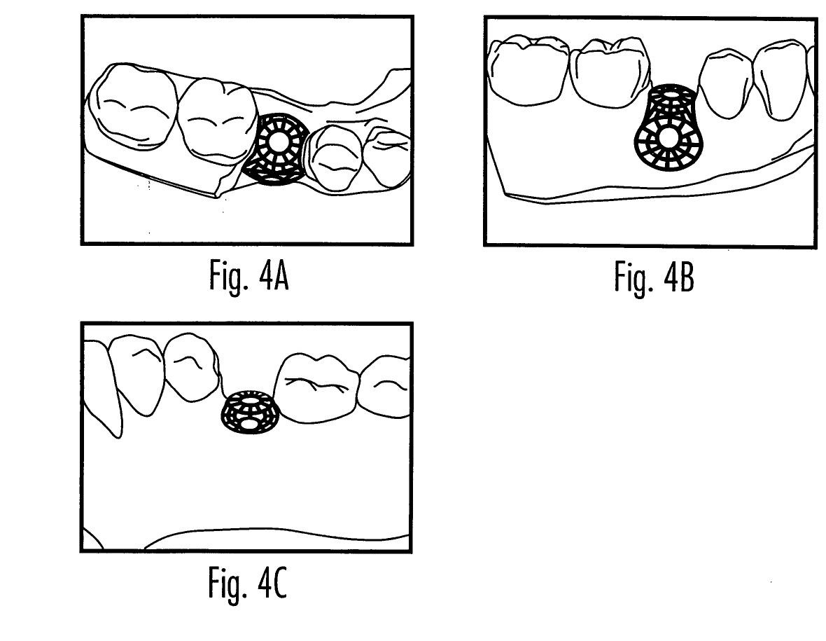

FIGS. 4A and 4B show, respectively, a top view of where, a device, such as device 10, would fit into a lower jaw for use in regenerating bone. A device according to the invention is not limited to any particular dimension and may be of any dimension suitable for use in a jaw bone. For example, a device may be dimensioned for use in the area that multiple teeth had occupied, or dimensioned to fit the entire lower jaw or upper jaw. Additionally, a device according to the invention could be preformed (which is preferred) to fit an area of the jaw, or could be formed by the dentist just prior to use.

FIG. 4D shows an impression of a lower jaw and where a device according to the invention would be placed to assist in reconstructing bone. In this Figure, the device would be 2.5×5 millimeters. FIG. 4E shows the back of the impression of FIG. 4D and FIG. 4F shows the top of the impression of FIG. 4D.

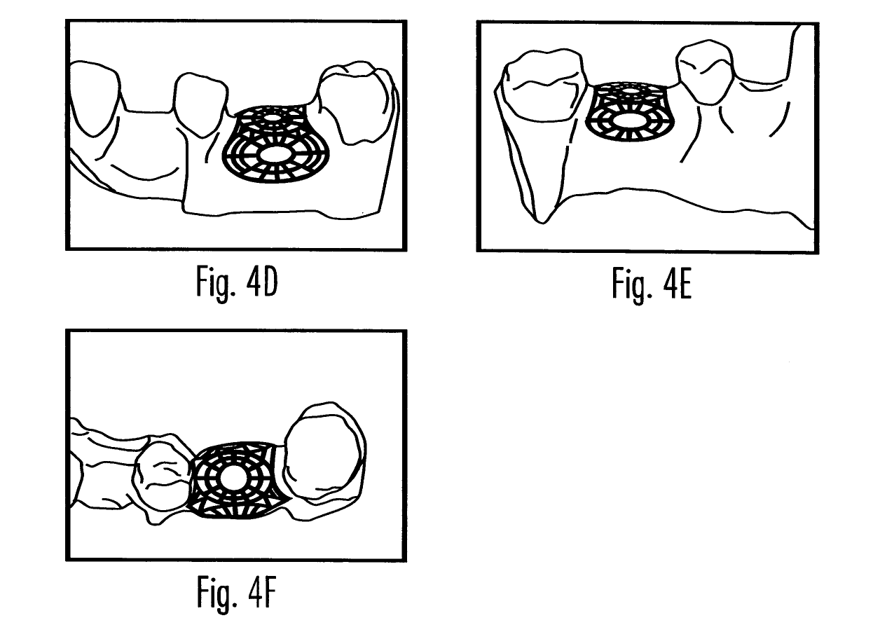

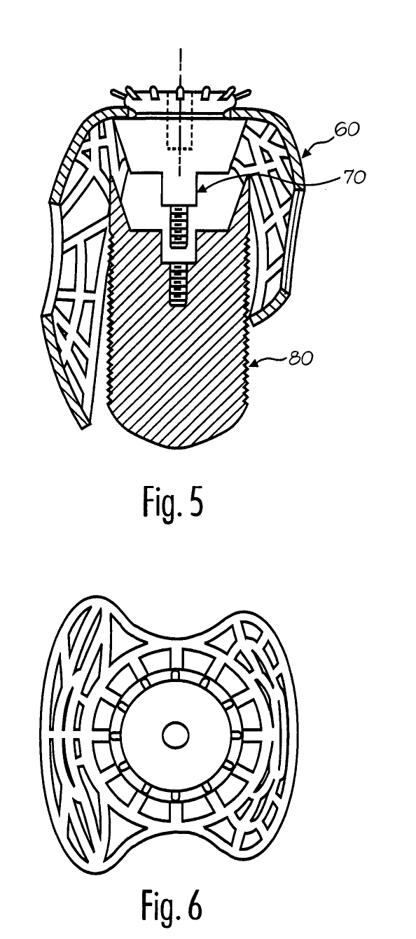

FIGS. 5 and 6 show a device 60 according to the invention premounted onto a mating component 70, which is received in a post 80. Any suitable technique may be used, however, for mounting a device according to the invention next to a bone. In this embodiment, and as shown in FIG. 5, a post 80 is provided for placing into a jaw bone using known methods. Device 60, which is a device according to the invention, is preformed using any suitable technique onto a mating component 70. Component 70 is for mating with post 80 and preferably for receiving a screw (not shown) or other attachment structure and allowing the structure to turn without turning device 60. Mating component 70 may or may not include a screw or other structure, premounted in it. Preferably, component 70 has a sleeve or collar 72 into which a screw passes and which facilitates turning of the screw without turning device 60.

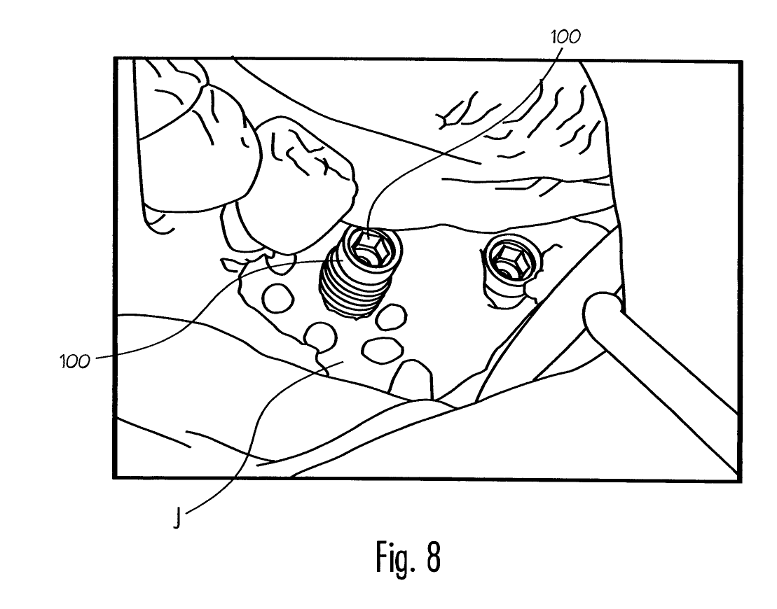

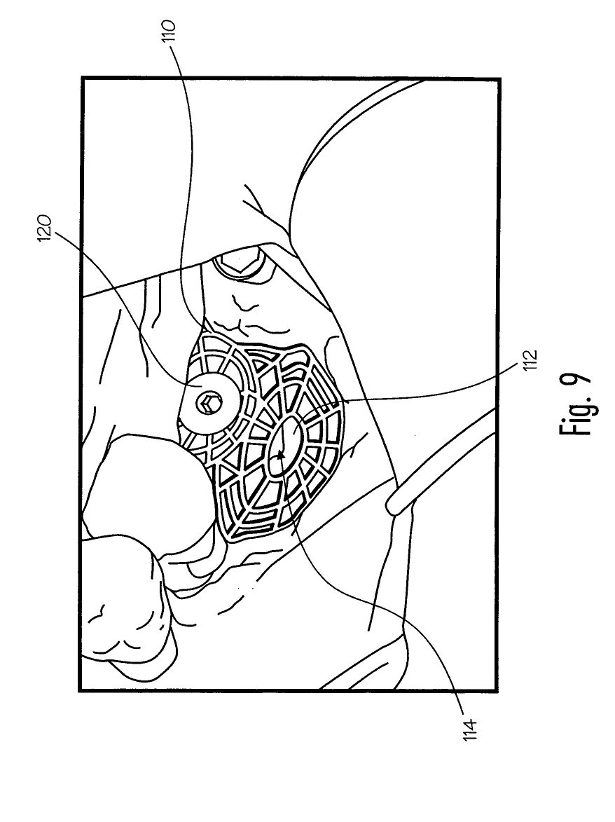

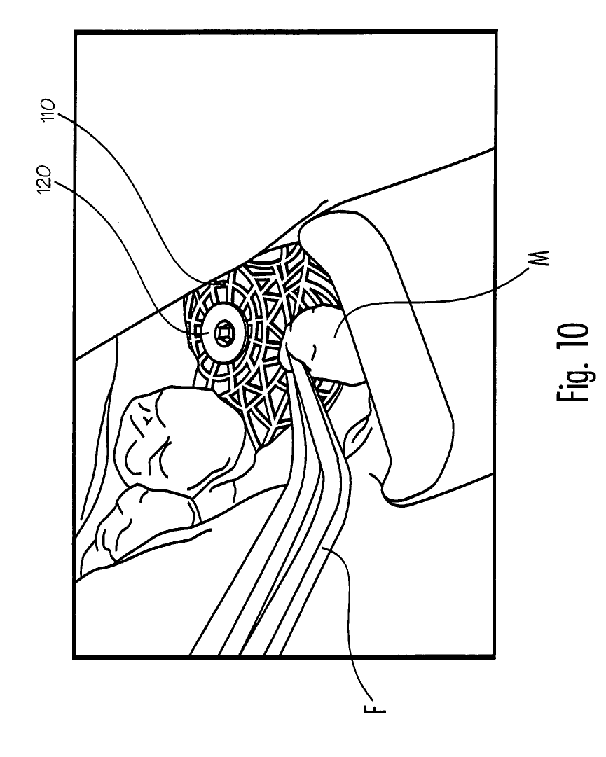

FIG. 8 shows an actual jaw bone J having posts 100 positioned therein. FIG. 9 shows a device 110 according to the invention having at least one opening 112 being mounted on one of posts 100 by a screw 120. FIG. 10 shows a material M (preferably a collagen sponge) saturated with morphogenetic protein (preferably BMP) being inserted by forceps F into opening 112 of device 110 to be retained in cavity 114 next to jaw bone J.

Having described preferred embodiments of the invention, variations that do not depart from the spirit of the invention may occur to others. The invention is thus not limited to the preferred embodiments but is instead set forth in the following claims and legal equivalents thereof.

1. A device to assist in regenerating bone, the device configured to at least partially surround the bone to be regenerated and defining a cavity next to the bone, the device further including one or more openings through which a material may be placed into the cavity, the cavity for retaining the material next to the bone, the material comprising a substance for stimulating bone growth.

2. The device of claim 1 that is comprised of wire mesh.

3. The device of claim 1 that is preformed to fit over the bone to be regenerated.

4. The device of claim 1 wherein the bone is a jaw bone of a human.

5. The device of claim 1 wherein each of the openings has a dimension of about 0.180 inches by 0.090 inches.

6. The device of claim 1 wherein there are two openings.

7. The device of claim 6 wherein the device has two sides and a top opening is on a side of the device and the other opening is on the opposite side of the device.

8. The device of claim 2 wherein the wire mesh is comprised of steel.

9. The device of claim 1 that further includes a space through which the shaft of a screw may pass so that the device may be attached to a post by the screw.

10. The device of claim 9 that further includes a post retained in the bone, and a screw with a shaft, the shaft passing through the spaces and being received in the post thereby securing the device to the post.

11. The device of claim 1 that is dimensioned to fit over the entire lower jaw of a human.

12. The device of claim 1 that is dimensioned to fit over the entire upper jaw of a human.

13. The device of claim 1 wherein the material is a collagen sponge.

14. The device of claim 1 wherein the substance is morphogenetic protein.

15. The device of claim 13 wherein the sponge contains morphogenetic protein.

16. The device of claim 1 that snap fits onto a post.

17. The device of claim 1 that further includes a material retained in the cavity.

18. The device of claim 17 wherein the substance is morphogenetic protein.

19. The device of claim 17 wherein the material is positioned in the cavity prior to the device being secured to a post affixed in the bone.

20. The device of claim 2 wherein the wire mesh has a diameter of 0.015″.

21. The device of claim 1 that includes a flap, the flap movable to cover one of the openings.

22. The device of claim 21 wherein each of the openings includes a flap movable to cover the opening.

23. The device of claim 1 wherein the device is formed of a material that can be absorbed into the body.

24. The device of claim 1 that further includes a mating component, the mating component having a screw for attaching to a post.

25. The device of claim 24 wherein the mating component is configured to mate with the configuration of a post mounted in a jaw.

26. The device of claim 24 wherein the screw can turn without turning the device.
